SEQUENCE LISTING (5 sequences)

SEQ ID NO: 1 - RNA, 127 nt, Hepatitis B virus
ggaucccgaucuucugaugaguccgugaggacgaaacgaguuccaugcaggugcugaugaguccgugaggacgaaaccucugcgcccugcucugaugaguccgugaggacgaaacgugccugaauuc

SEQ ID NO: 2 - RNA, 63 nt, Unknown
  misc_RNA
cgauagcagacucuaaaucugccgucaucgacuucgaagguucgaauccuucccaggacacca

SEQ ID NO: 3 - RNA, 23 nt, Unknown
  misc_RNA
nnnnnnngnnnnnnunnnnnnnn

SEQ ID NO: 4 - RNA, 66 nt, Unknown
  misc_RNA
nnnnnnnagcagacucuaaaucugccgucaucgacuucgaagguucgaauccuucnnnnnncacca

SEQ ID NO: 5 - RNA, 49 nt, Unknown
  misc_RNA
nnnnnnnacgucaucgacuucgaagguucgaauccuucnnnnnncacca

(19) United States
(12) Patent Application Publication
(10) Pub. No.: US 2002/0102537 A1
(43) Pub. Date: Aug. 1, 2002

(54) Retroviral production

(76) Inventors: Alan John Kingsman, Oxford (GB); Jason Slingsby, London (GB); Melvyn Yap, London (GB)

Correspondence Address: FROMMER LAWRENCE & HAUG, 745 FIFTH AVENUE- 10TH FL., NEW YORK, NY, 10151, US

(21) Appl. No.: 10/001,220

(22) Filed: Nov. 15, 2001

Related U.S. Application Data
(63) Continuation-in-part of application No. PCT/GB00/01974, filed on May 22, 2000.

(30) Foreign Application Priority Data
May 21, 1999 (GB) 9911961.2
May 21, 1999 (GB) 9911812.7
May 24, 1999 (GB) 9911976.0

(51) Int. Cl. A61K 39/21; C12N 7/01; C07H 21/02; C12Q 1/70; C07H 21/04; C12N 7/00; C12N 15/00; C12N 15/09; C12N 15/63; C12N 15/70; C12N 15/74; C12N 5/00; C12N 5/02

(52) U.S. Cl. 435/5; 435/235.1; 435/325; 424/188.1; 424/208.1; 536/23.72; 435/320.1

(57) ABSTRACT

A method is provided for enhancing the production of an infectious retrovirus comprising an envelope polypeptide in a producer cell which method comprises inhibiting the expression or activity in the producer cell of an endogenous receptor which is capable of binding to the envelope polypeptide of said retroviruses.

### FIELD OF THE INVENTION

The present invention relates to a method for improving the packaging efficiency of retroviral vectors.

### BACKGROUND OF THE INVENTION

Retroviral technology has gained immense popularity in recent times for the stable delivery of genes into cells (for recent review, see Miller, 1997). The applications are widespread in the fields of medicine, where it is used to deliver therapeutic genes to rectify genetic disorders, and also in science generally, where it is used to introduce genes into cells so as to study their functions (Miller, 1997). One reason for the popularity of retroviruses is that they are far more efficient in introducing genes to cells when compared to conventional methods of transfection. This is because the genes are packaged into virions which contain envelope proteins that bind to receptors on the target cells. This process enhances the entry of the gene into the cell.

Retroviruses are presented with a paradox in their life cycle. Interaction between the viral envelope and the cell receptor enables the virus to enter the cell. However, the same interaction between receptors in the infected cell and the newly synthesised envelope proteins limits the pool of envelope available for virion incorporation. In complex retroviruses such as HIV, this problem is solved by the expression of the vpu gene product which down regulates the receptors on the infected cell (Jabbar, 1995). In other retroviral systems, mechanisms to prevent receptor-envelope interaction have not been described (Swanstrom and Wills, 1997).

### SUMMARY OF THE INVENTION

We have found, while investigating using a three plasmid transient transfection method (Soneoka et al., 1995) which components are limiting in retroviral production, that under conditions where none of the viral components were saturating, the viral envelope component was limiting when its cognate receptor was found on the producer cell.

Accordingly, to alleviate the limitation of envelope on viral production, it is an object of the present invention to down-regulate the receptors on producer cells so as to increase the amount of envelope available for incorporation into virions.

Thus, the present invention provides a method for enhancing the production of an infectious retrovirus in a producer cell which method comprises inhibiting the expression in the producer cell of an endogenous receptor which binds the envelope polypeptide of said retrovirus.

Preferably expression of the receptor is inhibited by expressing in the producer cell a gene product capable of binding to and effecting the cleavage, directly or indirectly, of a nucleotide sequence encoding the receptor, or a transcription product thereof.

Preferably the gene product is selected from a ribozyme, an anti-sense ribonucleic acid and an external guide sequence, more preferably a ribozyme.

In a preferred embodiment the infectious retroviruses produced by the producer cell are isolated for subsequent use.

The present invention also provides a composition comprising infectious retroviruses obtained by the method of the invention. Such compositions may be used in therapy.

The present invention further provides a method for producing a pharmaceutical composition which method comprises isolating the infectious retrovirus produced by the producer cell according to the method of the invention described above and admixing with a pharmaceutically acceptable carrier, diluent or excipient.

The present invention also provides a producer cell in which the capacity for producing an infectious retrovirus is enhanced by the method of the invention.

In a preferred embodiment, the present invention provide a nucleic acid comprising a nucleotide sequence encoding a ribozyme capable of binding to and effecting the cleavage of an RNA encoding a Pit2 receptor. Preferably, the nucleic acid comprises a nucleotide sequence as shown in FIG. 1 or a variant thereof capable of binding to and effecting the cleavage of an RNA encoding a Pit2 receptor.

### DETAILED DESCRIPTION OF THE INVENTION

Although in general the techniques mentioned herein are well known in the art, reference may be made in particular to Sambrook et al., Molecular Cloning, A Laboratory Manual (1989) and Ausubel et al, Current Protocols in Molecular Biology (1995), John Wiley & Sons, Inc.

Retroviruses

The retroviral vectors used in the production of infectious retroviruses according to the present invention may be derived from or may be derivable from any suitable retrovirus. A large number of different retroviruses have been identified. Examples include: murine leukemia virus (MLV), human immunodeficiency virus (HIV), simian immunodeficiency virus, human T-cell leukemia virus (HTLV). equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV). A detailed list of retroviruses may be found in Coffin et al., 1997, “Retroviruses”, Cold Spring Harbour Laboratory Press Eds: J M Coffin, S M Hughes, H E Varmius pp 758-763.

Details on the genomic structure of some retroviruses may be found in the art. By way of example, details on HIV and Mo-MLV may be found from the NCBI Genbank (Genome Accession Nos. AF033819 and AF033811, respectively).

The lentivirus group can be split even further into “primate” and “non-primate”. Examples of primate lentiviruses include human immunodeficiency virus (HIV), the causative agent of human auto-immunodeficiency syndrome (AIDS), and simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV). Preferably, where the lentivirus is HIV, the vpu gene product is excluded as the means of down-regulating receptor expression.

A distinction between the lentivirus family and other types of retroviruses is that lentiviruses have the capability to infect both dividing and non-dividing cells (Lewis et al1992; Lewis and Emerman 1994). In contrast, other retroviruses—such as MLV—are unable to infect non-dividing cells such as those that make up, for example, muscle, brain, lung and liver tissue.

Preferred vectors for use in the production of infectious retroviruses in accordance with the present invention are recombinant retroviral vectors, in particular recombinant lentiviral vectors, in particular minimal lentiviral vectors, teachings relating to which are disclosed in WO99/32646 and in WO98/17815.

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes.

Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a defective retroviral vector genome gag, pol and env may be absent or not functional. The R regions at both ends of the RNA are repeated sequences. U5 and U3 represent unique sequences at the 5′ and 3′ ends of the RNA genome respectively.

In a typical retroviral vector for use in gene therapy, at least part of one or more of the gag, pol and env protein coding regions essential for replication may be removed from the virus. This makes the retroviral vector replication-defective. The removed portions may even be replaced by a nucleotide sequence of interest (NOI), such as a nucleotide sequence encoding a therapeutic product, to generate a virus capable of integrating its genome into a host genome but wherein the modified viral genome is unable to propagate itself due to a lack of structural proteins. When integrated in the host genome, expression of the NOI occurs—resulting in, for example, a therapeutic and/or a diagnostic effect. Thus, the transfer of an NOI into a site of interest is typically achieved by: integrating the NOI into the recombinant viral vector; packaging the modified viral vector into a virion coat; and allowing transduction of a site of interest—such as a targeted cell or a targeted cell population.

A minimal retroviral genome for use in the present invention will therefore comprise (5′) R-U5—one or more first nucleotide sequences —U3-R (3′). However, the plasmid vector used to produce the retroviral genome within a host cell/packaging cell will also include transcriptional regulatory control sequences operably linked to the retroviral genome to direct transcription of the genome in a host cell/packaging cell. These regulatory sequences may be the natural sequences associated with the transcribed retroviral sequence. i.e. the 5′U3 region, or they may be a heterologous promoter such as another viral promoter, for example the CMV promoter.

Some retroviral genomes require additional sequences for efficient virus production. For example, in the case of HIV, rev and RRE sequence are preferably included. However the requirement for rev and RRE can be reduced or eliminated by codon optimisation.

Codon optimisation causes to an improvement in codon usage. By way of example, alterations to the coding sequences for viral components may improve the sequences for codon usage in the mammalian cells or other cells which are to act as the producer cells for retroviral vector particle production. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms.

The retroviral vector may be produced using a codon optimised gag and a codon optimised pol or a codon optimised env.

Accessory genes encode variety of accessory proteins capable of modulating various aspects of retroviral replication and infectivity. These proteins are discussed in Coffin et al, Chapters 6 and 7. Examples of accessory proteins in lentiviral vectors include but are not limited to tat, rev, nef, vpr, vpu, vif, vpx. An example of a lentiviral vector useful in the present invention is one which has all of the accessory genes removed except rev.

Once the retroviral vector genome is integrated into the genome of its target cell as proviral DNA, the nucleotide sequences of interest need to be expressed. In a retrovirus, the promoter is located in the 5′ LTR U3 region of the provirus. In retroviral vectors, the promoter driving expression of a therapeutic gene may be the native retroviral promoter in the 5′ U3 region, or an alternative promoter engineered into the vector. The alternative promoter may physically replace the 5′ U3 promoter native to the retrovirus, or it may be incorporated at a different place within the vector genome such as between the LTRs.

Thus, an NOI will also be operably linked to a transcriptional regulatory control sequence to allow transcription of the NOI to occur in the target cell. The control sequence will typically be active in mammalian cells. The control sequence may, for example, be a viral promoter such as the natural viral promoter or a CMV promoter or it may be a mammalian promoter. It is particularly preferred to use a promoter that is preferentially active in a particular cell type or tissue type in which the virus to be treated primarily infects. Thus, in one embodiment, a tissue-specific regulatory sequences may be used. The regulatory control sequences driving expression of the one or more first nucleotide sequences may be constitutive or regulated promoters. Another particularly preferred regulatory construct comprises an hypoxia responsive element, such as is described in WO99/15684, the contents of which are incorporated herein by reference.

Replication-defective retroviral vectors are typically propagated, for example to prepare suitable titres of the retroviral vector for subsequent transduction, by using a combination of a packaging or helper cell line and the recombinant vector. That is to say, that the three packaging proteins can be provided in trans (see below).

Producer cells

Retroviral producer cells are cells that contain all the elements necessary for the production of infectious recombinant retroviruses. These elements may be permanently present stably within the cell (for example integrated in the cell genome or in episomal form) and/or transiently provided, for example by transfection.

A packaging cell, by contrast, expresses one or more viral components required for packaging retroviral DNA but lacks a psi region. Packaging cell lines typically comprise one or more of the retroviral gag, pol and env genes. Thus, the packaging cell line produces the structural proteins required for packaging retroviral DNA but it cannot bring about encapsidation due to the lack of a psi region. However, when a recombinant vector carrying a defective viral genome comprising a psi region and typically a nucleotide sequence of interest (NOI) is introduced into the packaging cell line, the helper proteins can package the psi-positive recombinant vector to produce the recombinant virus stock. This virus stock can be used to transduce cells to introduce the NOI into the genome of the target cells. It is preferred to use a psi packaging signal, called psi plus, that contains additional sequences spanning from upstream of the splice donor to downstream of the gag start codon since this has been shown to increase viral titres.

The recombinant virus whose genome lacks all genes required to make viral proteins can tranduce only once and cannot propagate. These viral vectors which are only capable of a single round of transduction of target cells are known as replication defective vectors. Hence, the NOI is introduced into the host/target cell genome without the generation of potentially harmful retrovirus. A summary of the available packaging lines is presented in Coffin et al., 1997.

Packaging cell lines in which the gag, pol and env viral coding regions are carried on separate expression plasmids that are independently transfected into a packaging cell line are preferably used. This strategy, sometimes referred to as the three plasmid transfection method (Soneoka et al., 1995) reduces the potential for production of a replication-competent virus since three recombinant events are required for wild type viral production. As recombination is greatly facilitated by homology, reducing or eliminating homology between the genomes of the vector and the helper can also be used to reduce the problem of replication-competent helper virus production.

An alternative to stably transfected packaging cell lines is to use transiently transfected cell lines. Transient transfections may advantageously be used to measure levels of vector production when vectors are being developed. In this regard, transient transfection avoids the longer time required to generate stable vector-producing cell lines and may also be used if the vector or retroviral packaging components are toxic to cells. Components typically used to generate retroviral vectors include a plasmid encoding the gag/pol proteins, a plasmid encoding the env protein and a plasmid containing an NOI. Vector production involves transient transfection of one or more of these components into cells containing the other required components. If the vector encodes toxic genes or genes that interfere with the replication of the host cell, such as inhibitors of the cell cycle or genes that induce apotosis, it may be difficult to generate stable vector-producing cell lines, but transient transfection can be used to produce the vector before the cells die. Also, cell lines have been developed using transient transfection that produce vector titre levels that are comparable to the levels obtained from stable vector-producing cell lines.

Producer cells can be produced either from packaging cells by introducing into the packaging cell any remaining viral components required for infectious retrovirus production or they can be produced by introduction into a non-packaging cell, such as a 293T cell, of all the components required for infectious retrovirus production.

Producer cells/packaging cells can be of any suitable cell type. Most commonly, mammalian producer cells are used but other cells, such as insect cells are not excluded Clearly, the producer cells will need to be capable of efficiently translating the env and gag, pol mRNA. Many suitable producer/packaging cell lines are known in the art. The skilled person is also capable of making suitable packaging cell lines by, for example stably introducing a nucleotide construct encoding a packaging component into a cell line.

It is highly desirable to use high-titre virus preparations in both experimental and practical applications. Techniques for increasing viral titre include using a psi plus packaging signal as discussed above and concentration of viral stocks. In addition, the use of different envelope proteins, such as the G protein from vesicular-stomatitis virus has improved titres following concentration to 10⁹ per ml. However, typically the envelope protein will be chosen such that the viral particle will preferentially infect cells that are infected with the virus which it desired to treat. For example where an HIV vector is being used to treat HIV infection, the env protein used will be the HIV env protein.

Receptors and Retroviral envelope proteins

The endogenous receptor expressed by the producer cell, the expression and/or activity of which it is desired to reduce or inhibit, is able to bind the envelope protein of the infectious retrovirus. Our results indicate that the binding of the envelope protein to the receptor causes a reduction in the retroviral titre produced by the producer cell. Preferably the receptor is an amphotropic receptor and not an ectotropic receptor. A preferred receptor is Pit 2.

The retrovirus envelope protein may be the native envelope protein with respect to the recombinant retrovirus or it may be a different envelope protein if, for example, the retrovirus has been pseudo-typed, the process of producing a retroviral vector in which the envelope protein is not the native envelope of the retrovirus. Certain envelope proteins, such as MLV envelope protein and vesicular stomatitis virus G (VSV-G) protein, pseudotype retroviruses very well. Pseudotyping can be useful for altering the target cell range of the retrovirus. Alternatively, to maintain target cell specificity for target cells infected with the particular virus it is desired to treat, the envelope protein may be the same as that of the target virus, for example HIV. Preferably the envelope protein is an amphotropic envelope protein and not an ectotropic envelope protein.

Examples of endogenous receptors and viral envelope proteins that they bind are listed below:

| | |
|---|---|
| Retroviral Envelope protein | Receptor (human cells) |
| Simian type D (MPMV, SRV, | Na⁺ dependent neutral amino acid |
| Baev) Feline endogenous | transporter; widely expressed in human |
| RD114 Avian reticulo- | tissues and cell lines, including |
| endotheliosis viruses | haematopoietic cells. |
| MLV; amphotropic. 4070A. | Pit 1 and 2 |
| 10AI | |
| GALV | Pit 1 |
| FeL V-B | Pit 1 (+ Pit 2 for some) |
| Simian sarcoma associated | Pit 1 |
| virus | Na⁺ dependent phosphate transporters |
| HIV/SIV | CD4 and co-receptors, e.g. CXCR4, |
| | CCCR5 |
| Avian sarcoma leukosis | small, membrane associated protein. - 40 |
| viruses subgroup A | residue cysteine rich motif with homology |
| | to low density lipoprotein receptor |
| Avian sarcoma leukosis | Cell surface protein resembling receptor |
| viruses subgroups B. D | for certain cytokines, e.g. tumour necrosis |
| | factor |

Gene products for inhibiting receptor expression

Gene products for use according to the present invention which inhibit expression of an endogenous receptor may do so in several ways. They may interfere with receptor gene transcription, mRNA processing, mRNA stability, mRNA translation, post-translational processing and/or targeting to the relevant cell membrane. It may also be possible to inhibit the activity of functional receptor by providing a ligand which binds reversibly or irreversibly to the receptor, thus blocking its ability to bind retroviral envelope protein.

The gene product may be expressed in the producer cell by a variety of techniques known to the person skilled in the art. For example a nucleotide sequence encoding the gene product may be introduced into the producer cell. Preferably, the nucleotide sequence encoding the gene product is present in a viral vector, such as a retroviral vector. In particular, it may be possible to include one or more nucleotide sequences encoding gene products in the viral genome used to produce the infectious retrovirus.

It is particularly preferred to use gene products that are capable of effecting the cleavage and/or enzymatic degradation of a target nucleotide sequence, which will generally be a ribonucleotide encoding the receptor. As particular examples, ribozymes, external guide sequences and antisense sequences may be mentioned.

Ribozymes are RNA enzymes which cleave RNA at specific sites. Ribozymes can be engineered so as to be specific for any chosen sequence containing a ribozyme cleavage site. Thus, ribozymes can be engineered which have chosen recognition sites in transcribed viral sequences. By way of an example, ribozymes encoded by the first nucleotide sequence recognise and cleave essential elements of viral genomes required for the production of viral particles, such as packaging components. Thus, for retroviral genomes, such essential elements include the gag, pol and env gene products. A suitable ribozyme capable of recognising at least one of the gag, pol and env gene sequences, or more typically, the RNA sequences transcribed from these genes, is able to bind to and cleave such a sequence. This will reduce or prevent production of the gal, pol or env protein as appropriate and thus reduce or prevent the production of retroviral particles.

Ribozymes come in several forms, including hammerhead, hairpin and hepatitis delta antigenomic ribozymes. Preferred for use herein are hammerhead ribozymes, in part because of their relatively small size, because the sequence requirements for their target cleavage site are minimal and because they have been well characterised. The ribozymes most commonly used in research at present are hammerhead and hairpin ribozymes.

Each individual ribozyme has a motif which recognises and binds to a recognition site in the target RNA. This motif takes the form of one or more “binding arms”, generally two binding arms. The binding arms in hammerhead ribozymes are the flanking sequences Helix I and Helix III, which flank Helix II. These can be of variable length, usually between 6 to 10 nucleotides each, but can be shorter or longer. The length of the flanking sequences can affect the rate of cleavage. For example, it has been found that reducing the total number of nucleotides in the flanking sequences from 20 to 12 can increase the turnover rate of the ribozyme cleaving a HIV sequence, by 10-fold. A catalytic motif in the ribozyme Helix II in hammerhead ribozymes cleaves the target RNA at a site which is referred to as the cleavage site. Whether or not a ribozyme will cleave any given RNA is determined by the presence or absence of a recognition site for the ribozyme containing an appropriate cleavage site.

Each type of ribozyme recognises its own cleavage site. The hammerhead ribozyme cleavage site has the nucleotide base triplet GUX directly upstream where G is guanine, U is uracil and X is any nucleotide base. Hairpin ribozymes have a cleavage site of BCUGNYR, where B is any nucleotide base other than adenine, N is any nucleotide, Y is cytosine or thymine and R is guanine or adenine. Cleavage by hairpin ribozymes takes places between the G and the N in the cleavage site.

Multiple ribozymes can be included in series in a single vector and can function independently when expressed as a single RNA sequence. A single RNA containing two or more ribozymes having different target recognition sites may be referred to as a multitarget ribozyme. The placement of ribozymes in series has been demonstrated to enhance cleavage.

Antisense technology is well known on the art. There are various mechanisms by which antisense sequences are believed to inhibit gene expression. One mechanism by which antisense sequences are believed to function is the recruitment of the cellular protein RNAseH to the target sequence/antisense construct heteroduplex which results in cleavage and degradation of the heteroduplex. Thus the antisense construct, by contrast to ribozymes, can be said to lead indirectly to cleavage/degradation of the target sequence. Thus according to the present invention, a first nucleotide sequence may encode an antisense RNA that binds to either a gene encoding an essential/packaging component or the RNA transcribed from said gene such that expression of the gene is inhibited, for example as a result of RNAseH degradation of a resulting heteroduplex. It is not necessary for the antisense construct to encode the entire complementary sequence of the gene encoding an essential/packaging component—a portion may suffice. The skilled person will easily be able to determine how to design a suitable antisense construct.

External guide sequences (EGSs) are RNA sequences that bind to a complementary target sequence to form a loop in the target RNA sequence, the overall structure being a substrate for RNaseP-mediated cleavage of the target RNA sequence. The structure that forms when the EGS anneals to the target RNA is very similar to that found in a tRNA precursor. The the natural activity of RNaseP can be directed to cleave a target RNA by designing a suitable EGS. The general rules for EGS design are as follows, with reference to the generic EGSs shown in FIG. 2:

Rules for EGS design in mammalian cells (see FIG. 2)

Target sequence—All tRNA precursor molecules have a G immediately 3′ of the RNaseP cleavage site (i.e. the G forms a base pair with the C at the top of the acceptor stem prior to the ACCA sequence). In addition a U is found 8 nucleotides downstream in all tRNAs. (i.e. G at position I, U at position 8). A pyrimidine may be preferred 5′ of the cut site. No other specific target sequences are generally required.

EGS sequence—A 7 nucleotide ‘acceptor stem’ analogue is optimal (5′ hybridising arm). A 4 nucleotide ‘D-stem’ analogue is preferred (3′ hybridising arm). Variation in this length may alter the reaction kinetics. This will be specific to each target site. A consensus ‘T-stem and loop’ analogue is essential. Minimal 5′ and 3′ non-pairing sequences are preferred to reduce the potential for undesired folding of the EGS RNA.

Deletion of the ‘anti-codon stem and loop’ analogue may be beneficial. Deletion of the variable loop can also be tolerated in vitro but an optimal replacement loop for the deletion of both has not been defined in vivo.

As with ribozymes, described below, it is preferred to use more than one EGS. Preferably, a plurality of EGSs is employed, together capable of cleaving gag, pol and env RNA of the native retrovirus at a plurality of sites. Multiple EGSs can be included in series in a single vector and can function independently when expressed as a single RNA sequence. A single RNA containing two or more EGSs having different target recognition sites may be referred to as a multitarget EGS.

Further guidance may be obtained by reference to, for example, Werner et al. (1997); Werner et al. (1998); Ma et al. (1998) and Kawa et al. (1998).

Therapeutic uses

The infectious retroviral particles may comprise one or more coding sequences encoding therapeutic products. Therapeutic products include, but are not limited to, cytokines, hormones, antibodies, immunoglobulin fusion proteins, enzymes, immune co-stimulatory molecules, anti-sense RNA, a transdominant negative mutant of a target protein, a toxin, a conditional toxin, an antigen, a single chain antibody, tumour suppresser protein and growth factors. When included, such coding sequences are operatively linked to a suitable promoter.

Preferably the viral particles are combined with a pharmaceutically acceptable carrier or diluent to produce a pharmaceutical composition. Thus, the present invention also provides a pharmaceutical composition for treating an individual, wherein the composition comprises a therapeutically effective amount of the viral particle of the present invention, together with a pharmaceutically acceptable carrier, diluent, excipient or adjuvant. The pharmaceutical composition may be for human or animal usage.

The choice of pharmaceutical carrier, excipient or diluent can be selected with regard to the intended route of administration and standard pharmaceutical practice. Suitable carriers and diluents include isotonic saline solutions, for example phosphate-buffered saline. The pharmaceutical compositions may comprise as—or in addition to—the carrier, excipient or diluent any suitable binder(s), lubricant(s), suspending agent(s), coating agent(s), solubilising agent(s), and other carrier agents that may aid or increase the viral entry into the target site (such as for example a lipid delivery system).

The pharmaceutical composition may be formulated for parenteral, intramuscular, intravenous, intracranial, subcutaneous, intraocular or transdermal administration.

Where appropriate, the pharmaceutical compositions can be administered by any one or more of: inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The amount of virus administered is typically in the range of from 10³ to 10¹⁰ pfu, preferably from 10⁵ to 10⁸ pfu, more preferably from 10⁶ to 10⁷ pfu. When injected, typically 1-10 μl of virus in a pharmaceutically acceptable suitable carrier or diluent is administered.

Where the therapeutic sequence is under the control of an inducible regulatory sequence, it may only be necessary to induce gene expression for the duration of the treatment. Once the condition has been treated, the inducer is removed and expression of the NOI is stopped. This will clearly have clinical advantages. Such a system may, for example, involve administering the antibiotic tetracycline, to activate gene expression via its effect on the tet repressor/VP16 fusion protein.

The invention will now be further described in the Examples which follow, which are intended as an illustration only and do not limit the scope of the invention.

### BRIEF DESCRIPTION OF THE DRAWINGS

FIG. 1—Graph of viral titres vs amount of DNA encoding viral components

FIG. 2—Western analysis of viral supernatants using Anti-p15 (gag). 300 ml of viral supernatant was pelleted and loaded in each lane. Lane 1: 0.1 μg of all three plasmids; Lane 2: 1 mg of gag-pol, 0.1 μg of genome and env; Lane 3: 1 μg of genome, 0.1 μg of gag-pol and env; Lane 4: 1 μg of env, 0.1 μg of gag-pol and genome; Lane 5: 1 μg of all three plasmids; Lane 6: 0.1 μg of gag-pol, 1 μg of genome and env.

FIG. 3—Scheme of experiment to determine if the negative effect of gag-pol on viral titres is due to interference by defective particles.

FIG. 4—Diagrammatic representation of a hypothesis for the envelope-dependent negative effect of gag/gag-pol on viral titres.

FIG. 5—Sequence (SEQ ID NO:1) and structure of riboram.

FIG. 6A—Design of external guided sequence (SEQ ID NO:2) and target RNA sequence (SEQ ID NO:3).

FIG. 6B—Design of external guided sequences (SEQ ID NO:4 and SEQ ID NO:5).

### EXAMPLES

### Example 1

Effect of each retroviral component on viral titres.

We investigated the limiting components in retroviral production using a transient transfection method. The genetic elements required to produce retroviral vectors capable of transducing cells were separated into three expression plasmids: one carrying the gag-pol gene, one carrying the envelope gene and the third bearing the packaging signal and lacZ gene that are flanked by the long terminal repeats (LTRs). Virions are produced when all three plasmids are transfected into 293T cells (Soneoka et al., 1995).

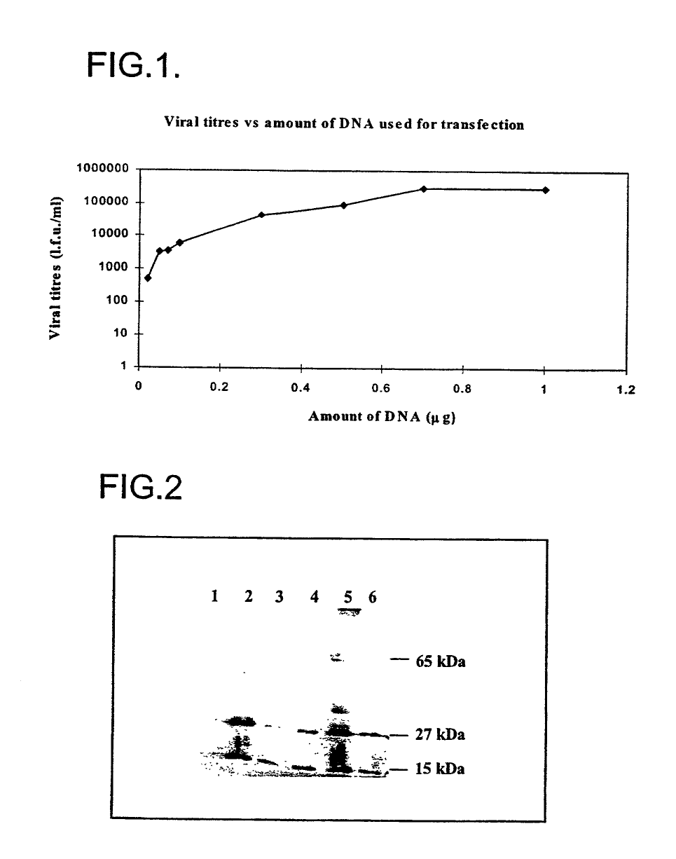

Firstly, we determined the conditions under which none of the three viral components are saturating. The results shown in FIG. 1 indicate that since none of the viral components are saturating at 0.1 μg of each plasmid, then 0.1 μg would be a suitable starting point from which the amount of each component could then be raised.

Raising the amount of one plasmid with respect to the other two, we measured the viral titres and compared them to the titres produced when equal amounts of all three plasmids were used.

TABLE 1

Amounts of plasmids (μg)^{a}

| | | Amphotropic | |
|---|---|---|---|
| gag-pol | Genome | Envelope | Titres (I.f.u./ml)^{b} |
| 0.1 | 0.1 | 0.1 | 6.5 ± 0.9 × 10³ |
| 1.0 | 0.1 | 0.1 | 1.6 ± 0 × 10³ |
| 0.1 | 1.0 | 0.1 | 4.1 ± 0.1 × 10⁴ |
| 0.1 | 0.1 | 1.0 | 1.9 ± 0.4 × 10⁴ |
| 1.0 | 1.0 | 1.0 | 3.5 ± 0.5 × 10⁵ |
| 0.1 | 1.0 | 1.0 | 1.6 ± 0.6 × 10⁵ |
| ^{a}Different amounts of plasmids were used to transfect 293T cells in 6 cm dishes using FuGene6 | | | |
| # transfection reagent (Boehringer Mannheim). | | | |
| ^{b}Viral titres (the number of infectious particles) were measured as the number of lacZ forming | | | |
| # units (I.f.u.) per ml as observed by X-gal staining. | | | |

It was found that 10-fold more gag/gal-pol as compared to genome and envelope reduced viral titres significantly (see Table 1). Therefore, gag/gag-pol had a negative effect on titres. The results in Table 1 also show that genome and env were limiting since titres could be raised by increasing the amounts of these two components during transfection. The negative effect of gag/gag-pol was only observed only under limiting conditions of env and genome.

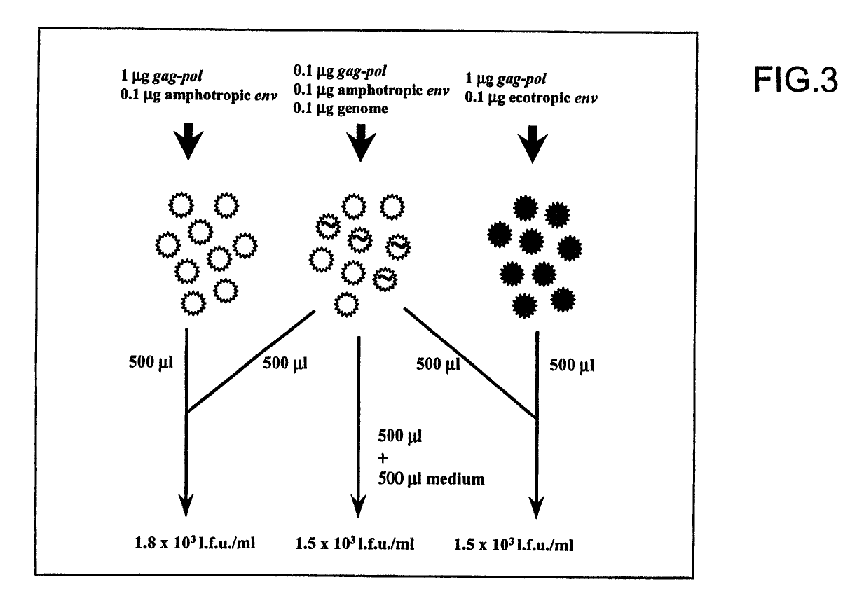

To investigate the total number of particles produced, a western blot analysis was carried out on the viral stocks produced as described in Table 1. This analysis showed that more particles were produced when more gag/gag-pol component was used (see FIG. 2, lanes 2 and 5). A large proportion of defective particles must be present in sample 2 since it had low viral titres despite having more particles.

### Example 2

The negative effect of gag-pol on viral titres is not due to interference by defective particles.

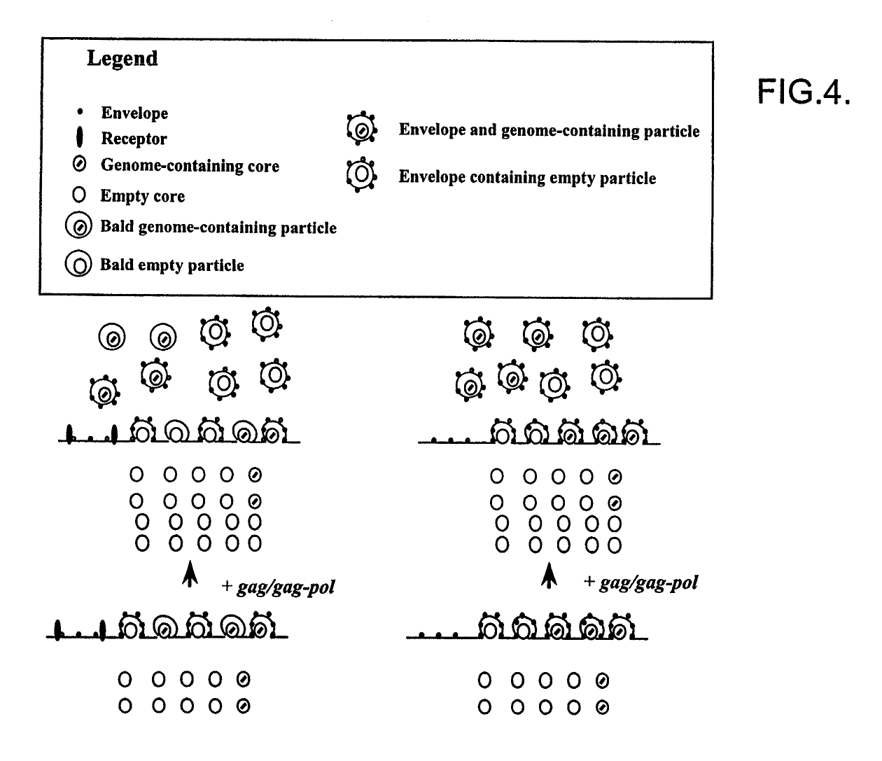

One explanation for the negative effect of gag/gag-pol on viral titres was that the defective particles were interfering with the binding of infectious particles. To test this hypothesis, an experiment using different types of envelope was carried out using the experimental approach shown in FIG. 3.

The results obtained showed that no decrease in titres was observed when either amphotropic or ecotropic empty particles were present in the viral stocks. Thus we conclude that the decrease in titres was not due to obstruction of receptors by enveloped defective particles.

### Example 3

The negative effects of gag/gag-pol can be cancelled by env or genome.

The negative effect of gag/gag-pol has been shown in Example 2 not to be due to an extracellular event. We therefore focused our attention on the intracellular events during viral production. To investigate if the negative effects of gag/gag-pol could be cancelled by env or genome, the following sets of transfections were performed and the following results obtained:

TABLE 2

Amounts of plasmids (μg)^{a}

| | Genome | Amphotropic | |
|---|---|---|---|
| gag-pol | (pHIT111) | Envelope | Titres (I.f.u./ml)^{b} |
| 0.1 | 0.1 | 0.1 | 6.5 ± 0.9 × 10³ |
| 1.0 | 0.1 | 0.1 | 1.6 ± 0 × 10³ |
| 0.1 | 1.0 | 0.1 | 4.1 ± 0.1 × 10⁴ |
| 1 | 1 | 0.1 | 1.3 ± 0.1 × 10⁴ |
| 0.1 | 0.1 | 1.0 | 1.9 ± 0.4 × 10⁴ |
| 1.0 | 0.1 | 1.0 | 1.3 ± 0.1 × 10⁴ |

These results show that titres do not decrease in the presence of excess gag/gag-pol when env or genome is not limiting. They also show that the negative effects of gag/gag-pol can be cancelled by env or genome.

### Example 4

The negative effect of gag/gag-pol is envelope-dependent

Since the amphotropic envelope was found to abolish the negative effect of gag/gag-pol on titres, an investigation was conducted to study the effects of other envelopes on viral titres.

It was found that with the ecotropic and VSV-G envelopes, env is saturating and there is no negative effect of gag/ag-pol on titres, whereas with the rabies and GALV envelopes, env is not saturating and there is a negative effect of gag/gag-pol on titres (see Table 3). We therefore conclude that the negative effect of gag/gag-pol is envelope dependent

These data imply that the effective concentration of env available for particle formation is less when GALV and Rabies are used compared to VSV-G and ecotropic. The most likely explanation is that GALV and Rabies envelopes are sequestered. This results in release of naked particles which are non-infectious, therefore effectively reducing the titres obtained.

TABLE 3

| Amounts | | | | | | |
|---|---|---|---|---|---|---|
| of plasmids (μg)^{a} | | | | | | |
| gag- | Gen- | | Titres (l.f.u/ml)^{b} | | | |
| pol | ome | Envelope | Ecotropic^{c} | VSV-G^{c} | Rabies^{d} | GALV^{e} |
| 0.1 | 0.1 | 0.1 | 5.0 × 10³ | 1.9 × 10³ | 1.3 × 10³ | 1.5 × 10³ |
| 1.0 | 0.1 | 0.1 | 5.2 × 10³ | 0.9 × 10³ | 4.4 × 10² | 5.0 × 10² |
| 0.1 | 1.0 | 0.1 | 1.2 × 10⁵ | 2.6 × 10⁴ | 2.2 × 10⁴ | 1.0 × 10⁴ |
| 0.1 | 0.1 | 1.0 | 8.0 × 10³ | 1.9 × 10⁴ | 9.0 × 10³ | 8.1 × 10³ |
| 1.0 | 1.0 | 1.0 | 2.5 × 10⁵ | 7.0 × 10⁴ | 1.7 × 10⁵ | 1.5 × 10⁵ |
| 0.1 | 1.0 | 1.0 | 1.4 × 10⁵ | 2.3 × 10⁴ | 2.5 × 10⁴ | 3.0 × 10⁴ |

^{a}Different amounts of plasmids were used to transfect 293T cells in 6 cm dishes using FuGene6 transfection reagent (Boehringer Mannheim).
^{b}Viral titres were measured as the number of lacZ forming units (l.f.u.) per ml as observed by X-gal staining.
^{c}Titrated on NIH3T3 cells.
^{d}Titrated on BHK21 cells.
^{e}Titrated on HT1080 cells.

### Example 5

Interaction of envelope with receptors limits its availability for incorporation into viral particles.

To test the hypothesis that interaction with receptors limits the availability of envelopes for incorporation into viral particles, the presence of receptors for the different envelopes in 293T cells was investigated indirectly by determination of titres using different envelopes.

TABLE 4

| Type of envelope | Titres (I.f.u./ml) |
|---|---|
| Amphotropic | 4.3 × 10⁵ |
| Ecotropic | 0 |
| Rabies-G | 7.0 × 10⁴ |
| VSV-G. | 4.0 × 10⁵ |

The results for 293T cells transduced with MoMLV based vectors pseudotyped with different envelopes are shown above in Table 4 (293T cells have been shown previously to be transduced by GALV pseudotyped particles (Eglitis et al., 1995)).

We therefore concluded that 293T producer cells express receptors for the amphotropic envelope, rabies-G, GALV and VSV-G but not receptors for the ecotropic envelope.

Summary of Examples 1-5

| | Receptors expressed | Negative effect of | Is envelope |
|---|---|---|---|
| Envelope | in 293T cells? | gag/gag-pol on titres? | limiting? |
| Amphotropic | Yes | Yes | Yes |
| Ecotropic | No | No | No |
| GALV | Yes | Yes | Yes |
| Rabies-G | Yes | Yes | Yes |
| VSV-G | Yes | No | No |

(i) The negative effect of gag/gag-pol on titres was observed only when envelope was not saturating. (ii) The envelope seemed to be limiting when its cognate receptor was expressed in the producer cell (293T) and not when it was absent.

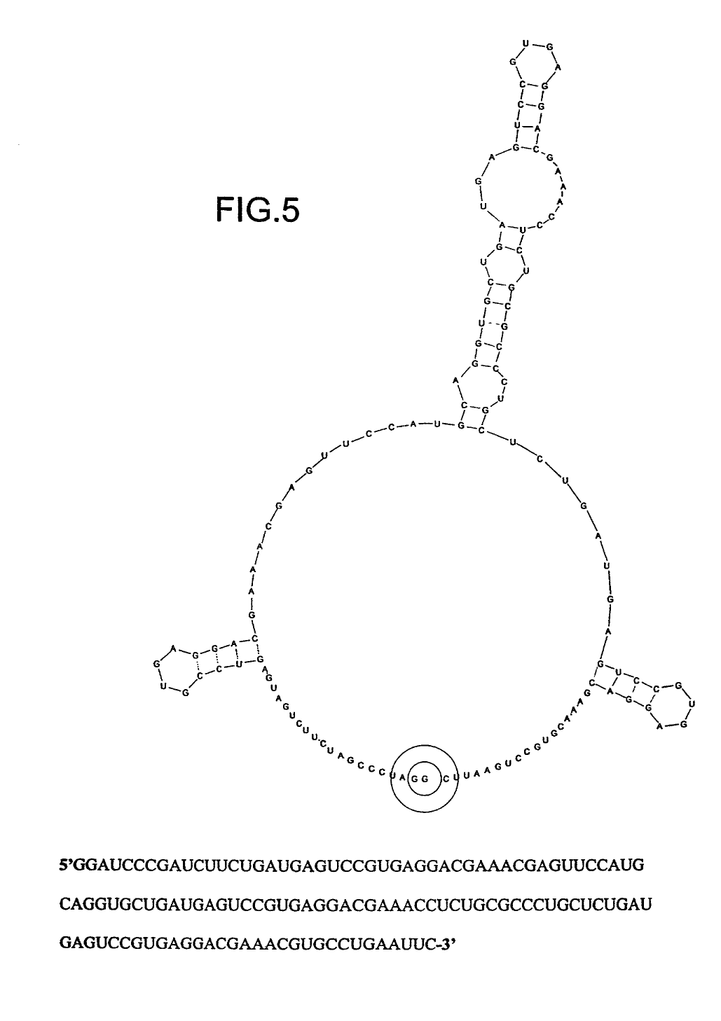

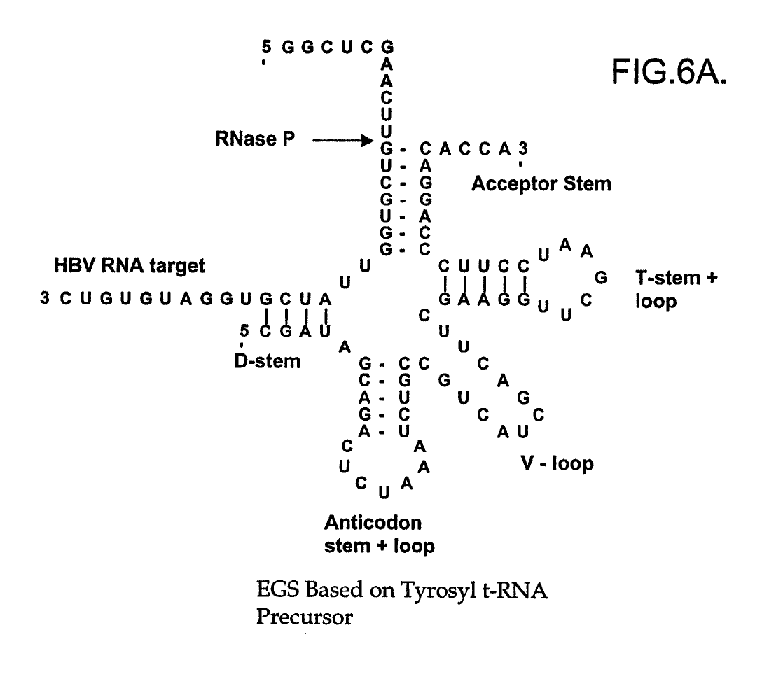

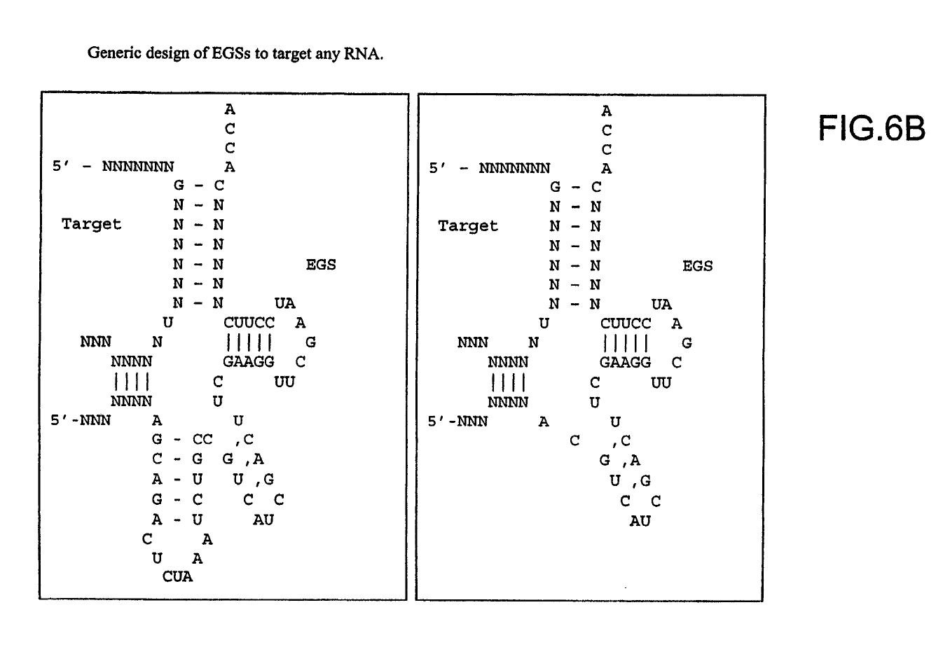

These data support the hypothesis that interaction with receptors can limit the availability of functional envelope (FIG. 4). Under this hypothesis, receptors interact with envelope and limit the pool of envelope available for incorporation into virions. Therefore is limiting. If excess gag/gag-pol is produced, then more empty cores are produced which compete with genome-containing cores for envelope during assembly. Hence there is a decrease in envelope and genome-containing particles, manifested as a decrease in titres.

VSV-G does not seem to conform with the hypothesis. The receptor of VSV-G is phosphatidylserine, a membrane phospholipid (Pal et al., 1987), which might not interact with the envelope on the membrane.

### Example 6

Down-regulation of the amphotropic receptor pit2 in producer cells

We have shown that under conditions where none of the retroviral component were saturating, the envelope component for amphotropic, GALV and rabies were limiting. One explanation is that interaction with the receptor limited the pool of envelope available for virion incorporation.

To test this hypothesis, a series of ribozymes directed against the human amphotropic receptor pit2 have been constructed. These are used to down-regulate the expression of pit2 in 293T and pCT6 cells and the effect on viral production is investigated.

Ribozyme design and construction

The mRNA of the amphotropic receptor pit2 is folded using the RNA draw programme. From the secondary structure of the RNA, two ribozymes are designed to target exposed regions while a third is designed to target the envelope-binding site. The order in which the three ribozymes is put together in a single construct is decided by folding the constructs using the RNA draw programme and selecting the one which has the least stable secondary structure (FIG. 1). This is to ensure that the binding of the ribozymes to the mRNA of pit2 is not obstructed by secondary structures within the ribozyme construct.

Two oligonucleotides corresponding to the sequence of the ribozymes and its complementary sequence flanked by BamHI and EcoRI restriction sites are synthesised. They are annealed and cloned into pBluescript. The resulting plasmid is designated pRiboram.

In vitro testing

The pRiboram is transcibed in vitro and the ribozyme is used to cleave the mRNA of pit2, which has also been transcribed in vitro. The cleaved products are detected on an agarose gel, indicating the ribozyme can function in vitro.

In vivo testing

The ribozyme construct is then sub-cloned from pRiboram into the BglII-EcoRI site of pSA91. which is a mammalian expression vector driven by the hCMV promoter. This plasmid is designated pCRiboram. pCRiboram was co-transfected with different combinations of amounts of gag-pol, env and genome expression plasmids into 293T cells. It is found that amphotropic envelope is no longer limiting in transient 3-plasmid transfection production systems of vector.

Production pCT6 cells

We have analysed the production characteristics of the human retroviral packaging cell lines FLYA13 (Cosset et al., 1995) and TEFLYA (S. Chapel-Fernandez and F. -L. Cosset, unpublished, 1998; Derrington et al., 1999). Both cell lines produce high-titre, complement resistant MLV vectors. The two packaging cell lines were derived using the same gag/pol and env expression constructs pCeB and pAF (Cosset et al., 1995). FLYA13 cells are based on HT1080 human fibrosarcoma cells, whereas TEFLYA cells are based on TE671 human rhabdomyosarcoma cells. We have analysed the packaging cell lines with respect to end point titre, transduction efficiency, and relative expression levels of retroviral proteins. Both packaging cells produce retroviruses containing a therapeutic genome at >10⁶ lacZ forming units (lfu) per ml. The retroviral preparations were concentrated and the gene transfer efficiency of the preparations was also investigated.

The retroviral genome used in this study was designated OB80. Transcription of the full-length retroviral genome is directed by a 5′ CMV promoter. OB80 is based on the pLXSN vector (Miller and Rosman, 1989). The genome contains the cytochrome P450 2B6 gene cloned upstream of an EMCV internal ribosome entry site (IRES) with the E.coli β-galactosidase marker gene (lacZ) cloned downstream of the IRES. An internal SV40 promoter directs expression of the neo^{r} gene.

A virus stock containing the OB80 genome was made in a transient expression system as previously described (Soneoka et al., 1995) using human 293 cells. The expression plasmid pRV67 (Kim et al., 1998) was used to pseudotype retroviral stocks with the VSV-G envelope protein. The retroviral genome was introduced into the packaging cell lines by retroviral transduction in the presence of 8 μg/ml polybrene (Sigma). VSV-G pseudotyped retrovirus was added to 50% confluent packaging cells at a low multiplicity of infection in 12 well plates. After 24 hours, the cells were split into 15 cm plates, and 1 mg/ml G418 (Life Technologies) was added to select for expression of the neo^{r} gene, transcribed from within the OB80 genome. After 14 days, high titre producer cell lines were identified by end point titration.

The retroviral genome OB80 was transduced into the TEFLYA packaging cell lines and producer cell lines were identified as above. Eight high titre TEFLYA lines were identified, and clone PCT6 was selected.

Production of a stable cell line expressing the ribozymes

PCT6 are transfected with pCRiboram using fugene and coselection with pIRESpuro (clontech). Drug resistant clones are selected and five of these are tested for increased vector production. All five give higher titres showing that reduced levels of cognate receptors increase retroviral vector titres.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

References

Cosset et al., 1995 J. Virol. 69: 7430-7436.

Derrington et al., 1999 Hum Gene Ther. 1999 May 1;10(7):1129-38.

Eglitis M. A., R. D. Schneiderman, P. M. Rice and M. V. Eiden. (1995). Evaluation of retroviral vectors based on the gibbon ape leukaemia virus. Gene Ther. 2(7): 486-92.

Hunter, E. (1997). Synthesis, assembly, and processing of viral proteins. Retroviruses. J. M. Coffin, S. H. Hughes and H. E. Varmus, Cold Spring Harbor Press: 263-334.

Jabbar, M. A. (1995). “The human immunodeficiency virus type 1 Vpu protein: Roles in virus release and CD4 downregulation.” Curr. Top. Microbiol. Immunol. 193: 107-120.

Kim et al., 1998 J. Virol., 72, 811-816

Kawa et al., 1998, RNA 4: 1397-1406.

Lewis et al1992 EMBO. J 11: 3053-3058.

Lewis and Emerman 1994 J. Virol. 68: 510-516.

Ma et al., 1998, Antisense and Nucleic Acid Drug Development 8: 415-426.

Miller, A. D. (1997). Development and applications of retroviral vectors. Retroviruses. J. M. Coffin, S. H. Hughes and H. E. Varmus, Cold Spring Harbor Press: 437-473.

Miller and Rosman, 1989 Biotechnique 7: 980-990.

Pal, R. Y. Barenholz and R. R. Wagner. (1987). Vesicular stomatitis virus membrane proteins and their interactions with lipid bilayers. Biochemica et Biophysica Acta. 906: 175-193.

Soneoka, Y., P. M. Cannon, E. E. Ramsdale, J. C. Griffiths, G. Romano, S. M. Kingsman, and A. J. Kingsman. (1995). A transient three plasmid expression system for the production of high titer retroviral vectors. Nucleic Acids Res. 23:628-33.

Swanstrom, R. and J. W. Wills. (1997). Synthesis, Assembly and Processing of Viral Proteins. In: Retroviruses. CSHL Press

Werner et al., 1997, Nucleic Acids Symposium Series No. 36: 19-21.

Werner et al., 1998, RNA 4: 847-855.

Then invention will now be further described by the following numbered paragraphs:

1. A method for enhancing the production of an infectious retrovirus comprising an envelope polypeptide in a producer cell which method comprises inhibiting the expression or activity in the producer cell of an endogenous receptor which is capable of binding to the envelope polypeptide of said retroviruses.

2. A method according to paragraph 1, wherein the receptor is selected from Pit1, Pit2 and CD4 and its coreceptors.

3. A method according to paragraph 1 or 2, wherein the envelope polypeptide is an amphotropic envelope polypeptide.

4. A method according to any one of paragraphs 1 to 3, wherein the expression of the receptor is inhibited by expressing in the producer cell a gene product capable of binding to and effecting the cleavage, directly or indirectly, of a nucleotide sequence encoding the receptor, or a transcription product thereof.

5. A method according to paragraph 4, wherein the gene product is selected from a ribozyme, an anti-sense ribonucleic acid and an external guide sequence.

6. A method according to paragraph 4, wherein the gene product is expressed by a viral vector.

7. A method according to paragraph 6, wherein the viral vector is a retroviral vector.

8. A method according to paragraph 7, wherein the retroviral vector is a lentiviral vector.

9. A method according to any one of the preceding paragraphs wherein the retrovirus is a lentivirus.

10. A method according to any one of the preceding paragraphs which further comprises isolating the infectious retrovirus produced by the producer cell.

11. A composition comprising an infectious retrovirus obtained by the method of paragraph 10.

12. A composition according to paragraph 11 for use in therapy.

13. A method for producing a pharmaceutical composition which method comprises isolating an infectious retrovirus produced by the producer cell according to the method of any one of paragraphs 1 to 9 and admixing the isolated infectious retrovirus with a pharmaceutically acceptable carrier, diluent or excipient.

14. A nucleic acid comprising a nucleotide sequence encoding a ribozyme capable of binding to an effecting the cleavage of an RNA encoding a pit2 receptor.

15. A nucleic acid according to paragraph 14 comprising a nucleotide sequence as shown in FIG. 1 or a variant thereof capable of binding to an effecting the cleavage of an RNA encoding a pit2 receptor.

16. A producer cell in which the capacity for producing an infectious retrovirus is enhanced by a method according to any of paragraphs 1 to 9.

17. A producer cell in which the expression or activity of an endogenous receptor, capable of binding to the envelope polypeptide of a retrovirus, is inhibited.

18. A producer cell according to paragraph 17, which expresses a gene product capable of binding to and effecting the cleavage, directly or indirectly, of a nucleotide sequence encoding the endogenous receptor, or a transcription product thereof.

1. A method for enhancing the production of an infectious retrovirus comprising an envelope polypeptide in a producer cell which method comprises inhibiting the expression or activity in the producer cell of an endogenous receptor which is capable of binding to the envelope polypeptide of said retroviruses.

2. A method according to claim 1, wherein the receptor is selected from Pit1, Pit2 and CD4 and its coreceptors.

3. A method according to claim 1, wherein the envelope polypeptide is an amphotropic envelope polypeptide.

4. A method according to claim 1, wherein the expression of the receptor is inhibited by expressing in the producer cell a gene product capable of binding to and effecting the cleavage, directly or indirectly, of a nucleotide sequence encoding the receptor, or a transcription product thereof.

5. A method according to claim 4, wherein the gene product is selected from a ribozyme, an anti-sense ribonucleic acid and an external guide sequence.

6. A method according to claim 4, wherein the gene product is expressed by a viral vector.

7. A method according to claim 6, wherein the viral vector is a retroviral vector.

8. A method according to claim 7, wherein the retroviral vector is a lentiviral vector.

9. A method according to claim 1 wherein the retrovirus is a lentivirus.

10. A method according to claim 1 which further comprises isolating the infectious retrovirus produced by the producer cell.

11. A composition comprising an infectious retrovirus obtained by the method of claim 10.

12. A composition according to claim 11 for use in therapy.

13. A method for producing a pharmaceutical composition which method comprises isolating an infectious retrovirus produced by the producer cell according to the method of claim 1 and admixing the isolated infectious retrovirus with a pharmaceutically acceptable carrier, diluent or excipient.

14. A nucleic acid comprising a nucleotide sequence encoding a ribozyme capable of binding to an effecting the cleavage of an RNA encoding a pit2 receptor.

15. A nucleic acid according to claim 14 comprising a nucleotide sequence as shown in FIG. 1 or a variant thereof capable of binding to an effecting the cleavage of an RNA encoding a pit2 receptor.

16. A producer cell in which the capacity for producing an infectious retrovirus is enhanced by a method according to claim 1.

17. A producer cell in which the expression or activity of an endogenous receptor, capable of binding to the envelope polypeptide of a retrovirus, is inhibited.

18. A producer cell according to claim 17, which expresses a gene product capable of binding to and effecting the cleavage, directly or indirectly, of a nucleotide sequence encoding the endogenous receptor, or a transcription product thereof.
